(19) United States
(12) Patent Application Publication
(10) Pub. No.: US 2015/0265412 A1
(43) Pub. Date: Sep. 24, 2015

(54) DRIVE DEVICE

(71) Applicant: Seiko Epson Corporation, Tokyo (JP)

(72) Inventor: Yutaka ARAKAWA, Hara (JP)

(21) Appl. No.: 14/643,223

(22) Filed: Mar. 10, 2015

(30) Foreign Application Priority Data
Mar. 19, 2014 (JP) 2014-056093

(51) Int. Cl. A61F 2/42; A41D 19/00; A61F 5/01

(52) U.S. Cl. CPC A61F 2/4241; A61F 5/013; A41D 19/00; A61F 2002/5007

(57) ABSTRACT

A finger joint drive device includes a first base section disposed on a back of a hand, a second base section rotatable with respect to the first base section, a drive section adapted to rotate the second base section, and an attachment mechanism. Further, the attachment mechanism is provided with a first curved portion having contact with a palm of the hand in an attached state, and a second curved portion and a third curved portion adapted to connect the first curved portion to the first base section. The first curved portion is arranged along a life line in the attached state. Therefore, the finger joint drive device can stably be fixed to the hand, and the degree of freedom of the fingers can sufficiently ensured.

### BACKGROUND

1. Technical Field

The present invention relates to a drive device.

2. Related Art

In the past, there has been known a finger joint drive device, which is attached to a hand, and assists an action of a finger in the attached state, namely bends and stretches the joints of the finger (see, e.g., JP-A-2010-240285 (Document 1)). The finger joint drive device described in Document 1 is provided with an action-assisting glove, a driven section, a drive section, a linear member, a biosignal detection section, and a control unit, and has a glove-like overall shape. When using the action-assisting glove, the action-assisting glove is worn so as to cover the whole hand. In such a configuration, in order to wear the action-assisting glove on the hand, it is necessary to set the size of the action-assisting glove to be larger than the hand of the user.

However, in the finger joint drive device described in Document 1, there is formed a slight gap between the hand and the action-assisting glove in the worn state in which the action-assisting glove is worn on the hand. Therefore, there is a possibility that the action-assisting glove moves relatively to the hand as much as an amount of the gap described above. In other words, in the finger joint drive device described in Document 1, there is a possibility that the fixation of the action-assisting glove to the hand becomes insufficient.

### SUMMARY

An advantage of some aspects of the invention is to provide a drive device, which can stably be fixed to an object such as a hand when using the drive device while being attached to the object.

Such an advantage can be achieved by the following aspects of the invention.

### Application Example 1

A drive device according to an aspect of the invention includes a first member, a second member rotatably disposed to the first member, a rotating section adapted to rotate the second member, and a third member provided to one of the first member and the second member, and having a hook.

According to this configuration, in the case of disposing the drive device to the hand, it becomes possible to fix the drive device to the hand by hooking the drive device on the hand with the hook. Therefore, the drive device can stably be fixed to the hand compared to, for example, the action-assisting glove described in Document 1.

### Application Example 2

In the drive device according to the aspect of the invention, it is preferable that the rotating section has a vibrating section including a piezoelectric element, and a driven body having contact with the vibrating section and driven by the vibrating section.

According to this configuration, by using the piezoelectric element as the drive section, miniaturization becomes possible. Therefore, in the case of disposing the drive device to the hand, it becomes possible to dispose the drive section to the finger.

### Application Example 3

In the drive device according to the aspect of the invention, it is preferable that the hook has a first hook and a second hook.

According to this configuration, in the case of disposing the drive device to the hand, the drive device can more stably be fixed to the hand compared to the case of providing a single hook.

### Application Example 4

In the drive device according to the aspect of the invention, it is preferable that the first hook and the second hook are connected to each other in the third member.

According to this configuration, in the case of disposing the drive device to the hand, the drive device can more stably be fixed to the hand compared to the case in which the two hooks are not connected to each other.

### Application Example 5

In the drive device according to the aspect of the invention, it is preferable that the drive device is to be disposed to a hand, the first member is disposed on a back of the hand, the second member is disposed on a finger of the hand, the third member is provided to the first member, and the hook has a contact section adapted to have contact with a palm of the hand.

According to this configuration, in the case of disposing the drive device to the hand, it is possible to clip the hand between the contact section and the first member.

### Application Example 6

In the drive device according to the aspect of the invention, it is preferable that the hook has a part disposed between a thumb and an index finger of the hand.

According to this configuration, the degree of freedom of the fingers can effectively be enhanced.

### Application Example 7

In the drive device according to the aspect of the invention, it is preferable that the hook has a part disposed on an opposite side of the thumb of the hand to the index finger of the hand.

According to this configuration, the degree of freedom of the fingers can effectively be enhanced.

### Application Example 8

In the drive device according to the aspect of the invention, it is preferable that the hook has a first hook and a second hook, the first hook is disposed between a thumb and an index finger of the hand, and the second hook is disposed on an opposite side of the thumb of the hand to the index finger of the hand.

According to this configuration, the degree of freedom of the fingers can effectively be enhanced, and at the same time, the drive device can more stably be fixed to the hand.

### Application Example 9

In the drive device according to the aspect of the invention, it is preferable that the hook has a part disposed on an opposite side of a little finger of the hand to a third finger.

According to this configuration, the degree of freedom of the fingers can effectively be enhanced.

### Application Example 10

In the drive device according to the aspect of the invention, it is preferable that the third member has a part arranged along a life line of the hand.

When bending a finger, in particular the thumb, the life line is a part forming a “valley.” By arranging the contact section along the life line, it is possible to more effectively inhibit the attachment mechanism from hindering the action of the fingers. Therefore, in the attached state, the degree of freedom of the fingers can further be enhanced.

### Application Example 11

In the drive device according to the aspect of the invention, it is preferable that the third member has a part lower in modulus of longitudinal elasticity than the first member.

According to this configuration, the third member can effectively deflect to deform, and thus, the attached state can easily be achieved.

### Application Example 12

In the drive device according to the aspect of the invention, it is preferable that the third member is formed of a single continuous linear object.

According to this configuration, the third member can easily be manufactured.

### Application Example 13

In the drive device according to the aspect of the invention, it is preferable that the hook has a first hook and a second hook, and the first hook and the second hook are formed integrally with each other.

According to this configuration, the third member can easily be manufactured.

### BRIEF DESCRIPTION OF THE DRAWINGS

The invention will be described with reference to the accompanying drawings, wherein like numbers reference like elements.

FIG. 1A is a diagram (a diagram viewed from the back side of a hand) showing a use state of a drive device (a first embodiment) according to the invention, and FIG. 1B is a diagram (a diagram viewed from the palm side of the hand) showing the use state of the drive device shown in FIG. 1A.

FIG. 2 is a cross-sectional view along the A-A line in FIG. 1A.

FIG. 3 is a cross-sectional view showing a state in which the finger is further bent in the state shown in FIG. 2.

FIG. 4 is a plan view of a drive section provided to the drive device shown in FIGS. 1A and 1B.

FIGS. 5A through 5C are explanatory diagrams showing an operation principle of the drive section.

FIG. 6A is a diagram (a diagram viewed from the back side of a hand) showing a use state of a drive device (a second embodiment) according to the invention, and FIG. 6B is a diagram (a diagram viewed from the palm side of the hand) showing the use state of the drive device shown in FIG. 6A.

FIG. 7A is a diagram (a diagram viewed from the back side of a hand) showing a use state of a drive device (a third embodiment) according to the invention, and FIG. 7B is a diagram (a diagram viewed from the palm side of the hand) showing the use state of the drive device shown in FIG. 7A.

### DESCRIPTION OF EXEMPLARY EMBODIMENTS

A drive device according to the invention will hereinafter be explained in detail based on some preferred embodiments shown in the accompanying drawings.

### First Embodiment

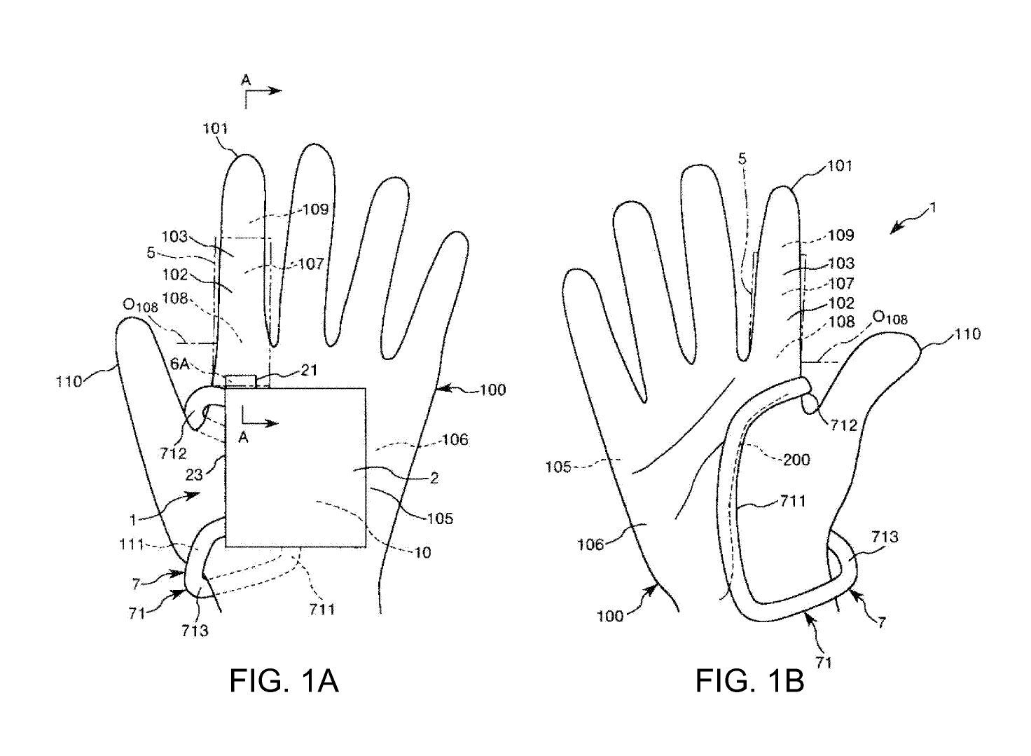

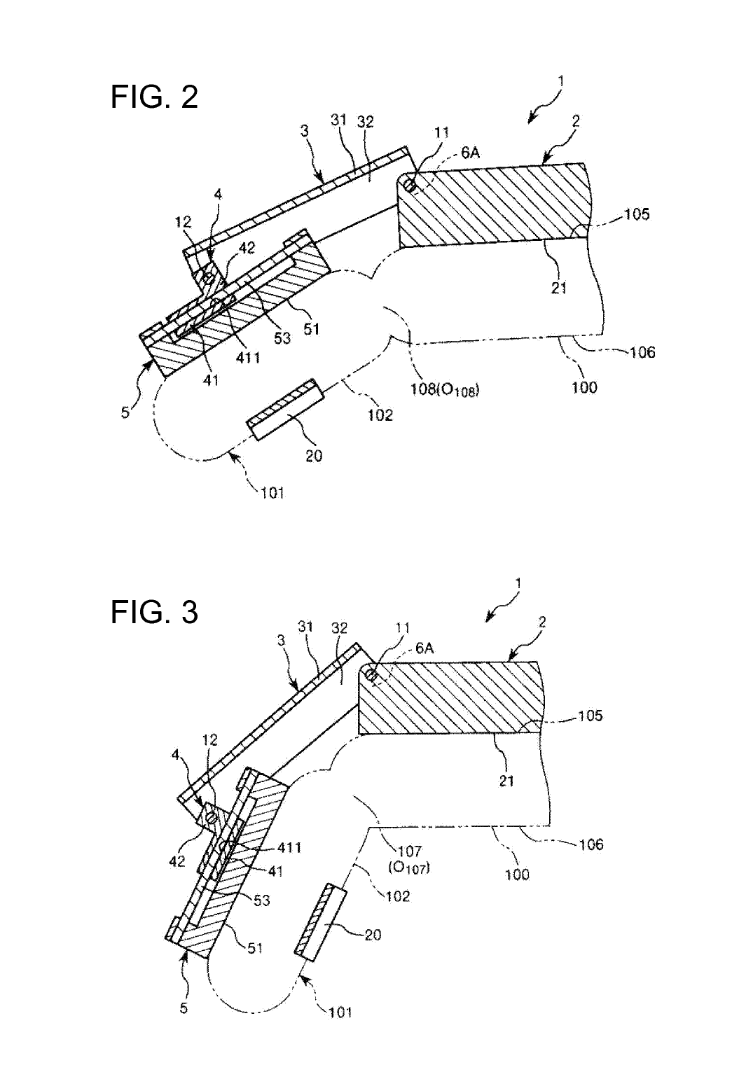

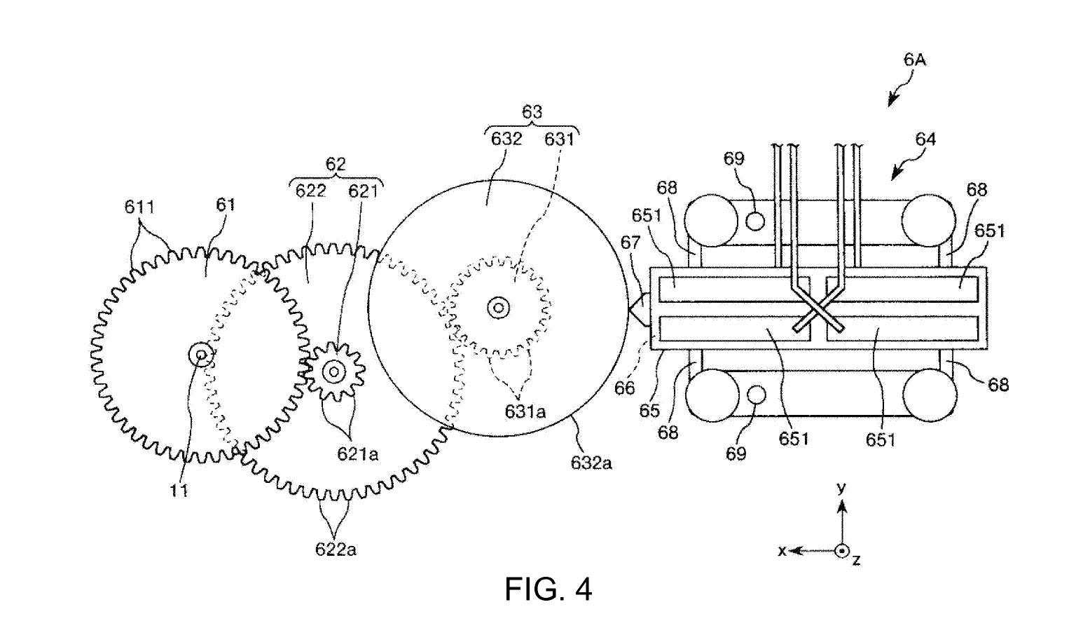

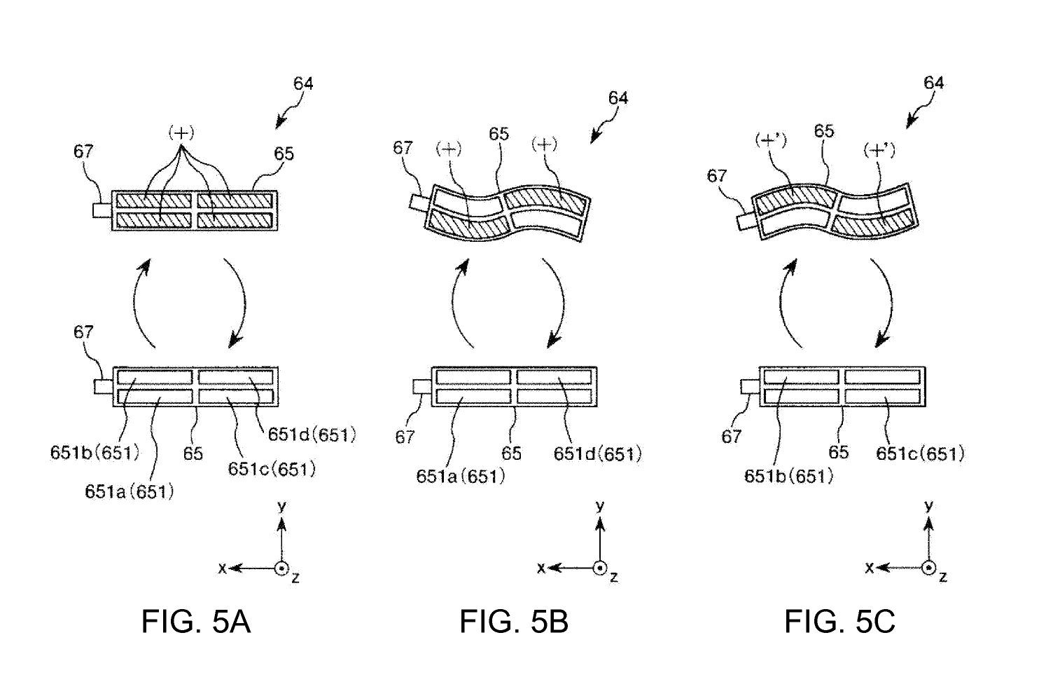

FIG. 1A is a diagram (a diagram viewed from the back side of a hand) showing a use state of the drive device (the first embodiment) according to the invention, and FIG. 1B is a diagram (a diagram viewed from the palm side of the hand) showing the use state of the drive device shown in FIG. 1A. FIG. 2 is a cross-sectional view along the A-A line in FIG. 1A. FIG. 3 is a cross-sectional view showing a state in which the finger is further bent in the state shown in FIG. 2. FIG. 4 is a plan view of a drive section provided to the drive device shown in FIGS. 1A and 1B. FIGS. 5A through 5C are explanatory diagrams showing the operation principle of the drive section.

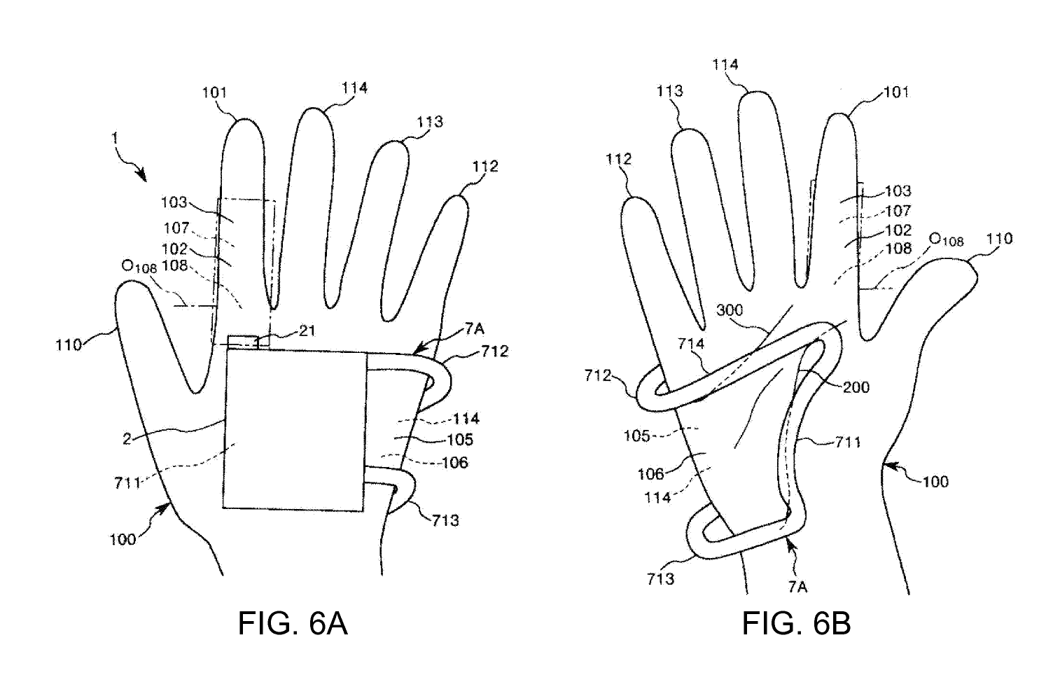

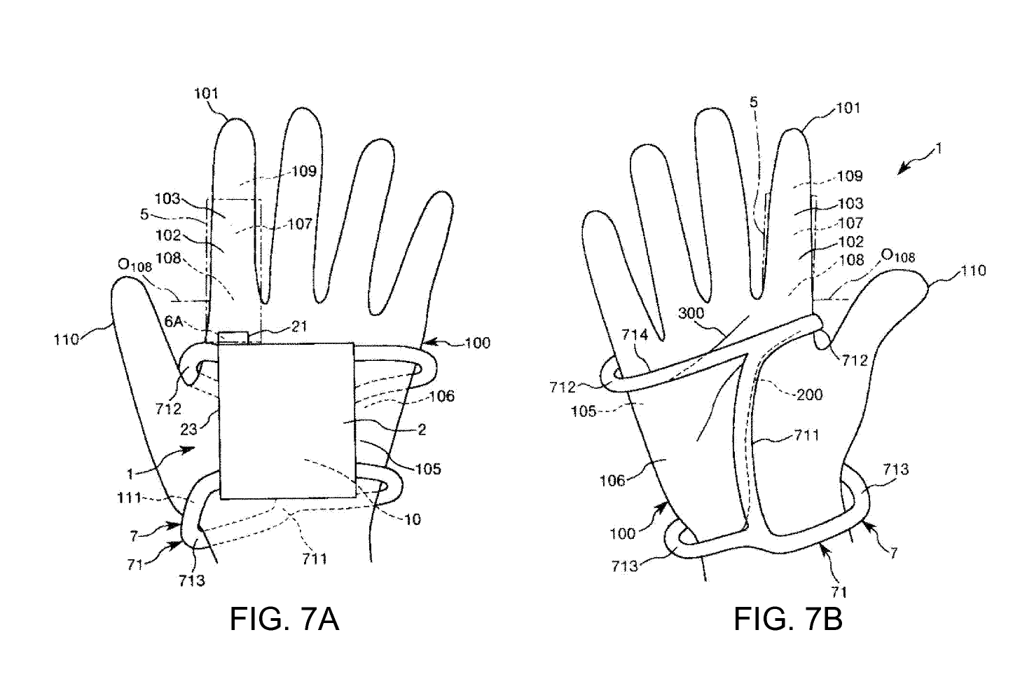

It should be noted that hereinafter the upper side in FIGS. 1A and 1B (the same applies to FIGS. 6A, 6B, 7A, and 7B) is referred to as a “fingertip side,” and an opposite side thereof is referred to as a “wrist side,” the left side in FIGS. 2 and 3 is referred to as the “fingertip side,” and the opposite side thereof is referred to as the “wrist side” for the sake of convenience of explanation. Further, the near side of the sheet in FIG. 4 is referred to as an “obverse side,” and an opposite side thereof is referred to as a “reverse side.”

A finger joint drive device (the drive device according to the invention) 1 shown in FIGS. 1A and 1B is attached to a hand 100 of a person who has a trouble in bending and stretching a finger due to, for example, an accident or a disease, a person weakened in hand grip, an older person who decreases in strength due to old age, or the like, namely to an index finger 101 in the present embodiment. Thus, the finger joint drive device is a device used for assisting bending and stretching, namely rotating the finger joint of the index finger 101 in the attached state. The finger joint drive device 1 is provided with a first base section (a first member) 2, a first link section 3, a second link section 4, a second base section (a second member) 5, and an attachment mechanism (a third member) 7. Further, the first base section 2, the first link section 3, the second link section 4, and the second base section 5 are sequentially connected in a direction from the wrist side toward the fingertip side. Hereinafter, configurations of the respective sections will be explained.

As shown in FIGS. 1A, 1B, 2, and 3, the first base section 2 is disposed on the back of the hand in the attached state. The first base section 2 is a member having an outer shape shaped like a flat block. Further, the first base section 2 incorporates a control section 10 described later, a button battery, and so on not shown.

Further, the thickness of the first base section 2 is not particularly limited, but is preferably in a range of, for example, about 2 through 20 mm, and is more preferably in a range of 5 through 10 mm. Thus, it can be reduced to apply a restriction to the action of the hand of the user, and at the same time, sufficient mechanical strength can be ensured.

As shown in FIGS. 1A and 1B (the same applies to FIGS. 2 and 3), the second base section 5 is disposed closer to the fingertip than the first base section 2, namely on the back-of-hand 105 side of a proximal phalanx 102 of the index finger 101, in the attached state. Thus, a metacarpophalangeal joint (a third joint) 108 located between the proximal phalanx 102 of the index finger 101 and the back-of-hand 105 can be bent and stretched using the finger joint drive device 1 as described later (see FIGS. 2 and 3).

The second base section 5 is a member having an outer shape shaped like a flat block. Further, it is preferable for a surface 51 having contact with the proximal phalanx 102 of the second base section 5 to be curved along the shape of a middle phalanx 103. Thus, the second base section 5 can be disposed on the proximal phalanx 102 without causing an uncomfortable feeling in the user of the finger joint drive device 1. Further, it results that the second base section 5 is stably disposed to the proximal phalanx 102.

Further, the second base section 5 is attached to the proximal phalanx 102 of the index finger 101 via an attachment band 20. The attachment band 20 is formed of a band member with an adjustable length, and has end portions respectively fixed to side surfaces of the second base section 5. The attachment band 20 can warp around the proximal phalanx 102 of the index finger 101 to the palm 106 to thereby make the second base section 5 adhere to the proximal phalanx 102. Thus, it is possible to prevent the second base section 5 from breaking away from the proximal phalanx 102.

As shown in FIGS. 1A and 1B, the first link section 3 is disposed on the fingertip side of the first base section 2. The first link section 3 is a member having a total length longer than the total length of the first base section 2 or the second base section 5. The first link section 3 has a top plate 31, and side walls 32 respectively projecting from both edge portions of the top plate 31. Further, the first base section 2 is pinched between the two side walls 32.

Further, each of the side walls 32 and a connection section 21 of the first base section 2 are connected to each other via a rotary support section 11. The rotary support section 11 is formed of a shaft provided to one of the side walls 32 and the first base section 2, and a bearing which is provided to the other thereof, and in which the shaft is inserted. Further, assuming a rotational axis O₁₀₈ around which the metacarpophalangeal joint 108 rotates due to bending and stretching actions, the rotational axis O₁₁ of the rotary support section 11 is parallel to the rotational axis O₁₀₈. Due to the rotary support section 11 having such a configuration, the first link section 3 can rotate around the rotational axis O₁₁ with respect to the first base section 2.

The second link section 4 is disposed on the fingertip side of the first link section 3. The second link section 4 has a sliding section 41 sliding with respect to the second base section 5, and a projecting section 42 projecting from the upper surface of the sliding section 41.

As shown in FIGS. 2 and 3, the sliding section 41 a part forming a cylindrical shape, namely having a hollow section 411, and a rail section 53 of the second base section 5 is inserted through the hollow section 411. It should be noted that the total length of the rail section 53 is sufficiently longer than the total length of the sliding section 41, and is preferably 1.5 through 3 times as long as the sliding section 41, and is more preferably 1.7 through 2.3 times as long as the sliding section 41, for example. By the sliding section 41 sliding while being guided by the rail section 53, the second base section 5 can relatively get closer to and away from the first base section 2. FIG. 2 shows the state in which the second base section 5 gets closer to the first base section 2, and FIG. 3 shows the state in which the second base section 5 gets away from the first base section 2.

The projecting section 42 is pinched by the two side walls 32 of the first link section 3. Further, the projecting section 42 is connected to the side walls 32 via a rotary support section 12. The rotary support section 12 is formed of a shaft provided to one of the projecting section 42 and the side walls 32, and a bearing which is provided to the other thereof, and in which the shaft is inserted. Further, the rotational axis O₁₂ of the rotary support section 12 is parallel to the rotational axis O₁₀₈. Due to the rotary support section 12 having such a configuration, the second link section 4 can rotate around the rotational axis O₁₂ parallel to the rotational axis O₁₀₈ similarly to the first link section 3. Since the rotational axis O₁₁ and the rotational axis O₁₂ are parallel to the rotational axis O₁₀₈, it is possible to bend and stretch the metacarpophalangeal joint 108 with the finger joint drive device 1 with ease, namely while preventing an unbearable force from acting on the metacarpophalangeal joint 108.

The constituent materials of the first base section 2, the first link section 3, the second link section 4, and the second base section 5 are not particularly limited, but a variety of resin materials such as polyethylene, and a variety of metal materials such as aluminum can be used. Further, the constituent material of the attachment band 20 is not particularly limited, but a variety of types of rubber materials such as silicon rubber can be used.

Further, as shown in FIGS. 1A and 1B, the finger joint drive device 1 is further provided with a drive section (a rotary section) 6A and the control section 10.

The drive section 6A is a mechanism section in charge of the rotational drive of the first link section 3 rotating via the rotary support section 11.

As shown in FIG. 4, the drive section 6A has a first rotor 61 concentrically coupled to the shaft of the rotary support section 11, a second rotor 62 for rotating the first rotor 61, a third rotor 63 for rotating the second rotor 62, and a piezoelectric motor 64 as a vibrating section for rotating the third rotor 63.

The first rotor 61 is a gear wheel having a disk-like shape, and having gear teeth 611 disposed in the outer circumferential edge thereof.

The second rotor 62 has a small gear wheel 621 and a large gear wheel 622. The small gear wheel 621 has gear teeth 621a engaging with the gear teeth 611 of the first rotor 61. The large gear wheel 622 is a gear wheel larger in base diameter than the small gear wheel 621. The large gear wheel 622 is disposed concentrically with the small gear wheel 621, and is coupled (fixed) to the reverse side of the small gear wheel 621.

The third rotor 63 has a small gear wheel 631 and a large disk section 632. The small gear wheel 631 has gear teeth 631a engaging with gear teeth 622a of the large gear wheel 622. The large disk section 632 has a disk-like shape, and has a diameter larger than the base diameter of the small gear wheel 631. The large disk section 632 is disposed concentrically with the small gear wheel 631, and is coupled to the obverse side of the small gear wheel 631.

As shown in FIG. 4, the piezoelectric motor 64 has two piezoelectric elements (piezo elements) 65 each including a piezoelectric material exerting a piezoelectric effect, and formed to have a plate-like shape, and a shim plate 66 sandwiched and bonded between these piezoelectric elements 65, and formed of a flat metal plate. Hereinafter, a longitudinal direction of the piezoelectric motor 64 is referred to as an “x direction,” a width direction of the piezoelectric motor 64 perpendicular to the x direction is referred to as a “y direction,” and a thickness direction of the piezoelectric motor 64 perpendicular to the x direction and the y direction is referred to as a “z direction.”

In each of the piezoelectric elements 65, there are disposed four electrodes 651 for applying a voltage to the piezoelectric elements 65. These electrodes 651 are arranged in a 2×2 matrix on each of the piezoelectric element 65, and are electrically connected to a battery (not shown) such as a button battery as a power supply source.

Further, the shim plate 66 made of metal has not only a role of reinforcing each of the piezoelectric elements 65, but also a role as a common electrode for applying the voltages to the respective piezoelectric elements 65, and is connected to the ground.

In an end portion in the x direction of the piezoelectric motor 64, there is disposed a projection section 67. The projection section 67 is formed integrally with the shim plate 66.

On both of the side surfaces facing to the y direction of the piezoelectric motor 64, there are disposed four support sections 68 for supporting the piezoelectric motor 64 in the state of being biased toward the side provided with the projection section 67. These support sections 68 are formed integrally with the shim plate 66, and are disposed on the four corners of the shim plate 66 having a rectangular shape. It should be noted that the support sections 68 adjacent to each other in the x direction are preferably coupled to each other via a coupling plate 69.

The operation principle of the piezoelectric motor 64 having such a configuration will be explained with reference to FIGS. 5A through 5C.

The piezoelectric motor 64 operates in accordance with an elliptic motion of the projection section 67 of the piezoelectric motor 64 made when applying the voltages to the electrodes 651 of each of the piezoelectric elements 65 at a constant frequency. The reason that the projection section 67 of the piezoelectric motor 64 makes the elliptic motion is as follows. It should be noted that since the electrodes 651 provided to each of the piezoelectric elements 65 are identical to each other except the fact that the arrangement places are different from each other, the electrodes 651 of the piezoelectric element 65 on the obverse side will be explained as representatives thereof.

Firstly, as well known to the public, the piezoelectric element 65 including the piezoelectric material has a property of expanding in response to application of a positive voltage. Therefore, as shown in FIG. 5A, by repeating an operation of applying a positive voltage to all of the four electrodes 651 and then removing the applied voltage at a predetermined frequency, the piezoelectric motor 64 (the piezoelectric element 65) can generate a kind of resonant phenomenon that the piezoelectric motor 64 expands and contracts in the x direction. It should be noted that the action of the piezoelectric motor 64 repeating the expansion and contraction in the x direction is referred to as a “stretching vibration,” and the directions (the ±x directions in the drawing) in which the piezoelectric motor 64 expands and contracts are referred to as “stretching directions.”

Further, as shown in FIG. 5B or 5C, by applying a voltage at a specific frequency to a set (a set of the electrode 651a and the electrode 651d, or a set of the electrode 651b and the electrode 651c) of two of the electrodes 651 located at diagonal positions, the piezoelectric motor 64 (the piezoelectric element 65) can generate a kind of resonant phenomenon that a tip portion (a portion provided with the projection section 67) in the x direction yaws in a vertical direction (the y direction) on the drawing. For example, as shown in FIG. 5B, when applying a positive voltage at a constant frequency to the set of the electrode 651a and the electrode 651d, the piezoelectric motor 64 repeats an action that the tip portion in the x direction moves downward. Further, as shown in FIG. 5C, when applying a positive voltage at a constant frequency to the set of the electrode 651b and the electrode 651c, the piezoelectric motor 64 repeats an action that the tip portion in the x direction moves upward. Such actions of the piezoelectric motor 64 are referred to as a “flexural vibration.” The directions (the ±y directions in the drawing) in which the piezoelectric motor 64 flexurally vibrates are hereinafter referred to as “flexion directions.”

Further, by appropriately selecting the physicality of the piezoelectric element 65 and the dimensions (the total length, the width, and the thickness) of the piezoelectric element 65, it is possible to cause the resonance in the “flexural vibration” to thereby excite the resonance in the “stretching vibration” at the same time. As a result, in the case of applying the voltage to the set of the electrode 651a and the electrode 651d in such a manner as shown in FIG. 5B, the tip portion (the portion provided with the projection section 67) of the piezoelectric motor 64 performs such an action (an elliptic motion) as to draw an ellipse clockwise on the drawing. Further, in the case of applying the voltage to the set of the electrode 651b and the electrode 651c in such a manner as shown in FIG. 5C, the tip portion of the piezoelectric motor 64 performs an elliptic motion in a counterclockwise direction on the drawing. The same as in the case of the piezoelectric element 65 on the obverse side is completely true for the piezoelectric element 65 on the reverse side.

The piezoelectric motor 64 drives the first link section 3 as a driven body using such an elliptic motion.

Specifically, the piezoelectric motor 64 generates the elliptic motion in the state in which the projection section 67 of the piezoelectric motor 64 is pressed against the outer circumferential edge 632a of the large disk section 632 of the third rotor 63. Thus, the projection section 67 repeats the action of moving from left to right (or from right to left) in a state of being pressed against the driven body when the piezoelectric motor 64 expands, and returning to the initial position in a state of being separated from the driven body when the piezoelectric motor 64 contracts. As a result, the third rotor 63 rotates in one direction due to a frictional force applied by the projection section 67. Then, the rotational force is transferred via the small gear wheel 631 of the third rotor 63, the large gear wheel 622 and the small gear wheel 621 of the second rotor 62, and the first rotor 61 sequentially in this order. Thus, it is possible to rotationally drive the first link section 3 via the rotary support section 11.

As described above, in the finger joint drive device 1, the rotational drive of the first link section 3 can surely be performed with a simple configuration of using the piezoelectric elements 65. Further, the configuration of using the piezoelectric elements 65 also makes a contribution to miniaturization and height reduction of the finger joint drive device 1.

Further, in the piezoelectric motor 64, the further the metacarpophalangeal joint 108 of the index finger 101 bends in the gripping direction, the higher the resolution can be made, which is preferable.

It should be noted that the drive section 6A is in charge of the rotational drive of the first link section 3 in the present embodiment, but is not limited to this configuration, and can also be in charge of the rotational drive of the second link section 4. In this case, the rotational drive of the second link section 4 can also be performed in a reliable manner similarly to the case described above.

The control section 10 is for controlling the operation of the drive section 6A based on a program stored in advance. The control section 10 is incorporated in the first base section 2 together with the battery (not shown) such as a button battery for supplying electrical power to the drive section 6A. It should be noted that the configuration of the control section 10 is not particularly limited, and a configuration having a microprocessor and a memory device, for example, can be adopted.

Then, the attachment mechanism 7 will be explained. As shown in FIGS. 1A and 1B, the attachment mechanism 7 is for keeping the attached state, namely for fixing the finger joint drive device 1 to the hand 100 in the attached state. The attachment mechanism 7 is formed of a hook 71 made of a linear object having a circular lateral cross-sectional shape. Further, the both end portions of the hook 71 are connected to respective positions of the first base section 2 different from each other. The hook 71 has a loop-like shape together with the first base section 2. In the present embodiment, the hook 71 has the both end portions are connected to the side surface 23 on the thumb 110 side of the first base section 2.

It should be noted that in the present embodiment, the “hook” denotes what functions as a hooking section capable of hooking an object such as a finger. Therefore, the shape of the “hook” is not particularly limited providing the shape has an inflection point, for example, a shape curved so as to have an arc-like shape such as a “U” shape, an “O” shape, or a “J” shape, or a shape bent in the middle.

Further, the hook 71 has elasticity. It should be noted that the hook 71 has such rigidity that the hook 71 can be bent by applying an external force, but does not deflect to deform under the own weight in the natural state with no external force applied.

Such a hook 71 can be divided into a first curved portion 711 disposed on the palm 106 in the attached state, a second curved portion (a first hook) 712, and a third curved portion (a second hook) 713.

The first curved portion 711 forms a contact section having contact with the palm 106 in the attached state. The first curved portion 711 extends from the wrist side to the fingertip side so as to follow a life line 200 as close as possible, and curves so as to slightly be oblique to the vertical direction in FIGS. 1A and 1B in the attached state.

Further, the first curved portion 711 is disposed so as to overlap the first base section 2 in a planar view of the first base section 2. Thus, it results that in the attached state, the first curved portion 711 and the first base section 2 are opposed to each other via the hand 100. Therefore, the first curved portion 711 can clip the hand 100 in the thickness direction of the hand 100 together with the first base section 2 in the attached state. Therefore, it is possible to stably fix the finger joint drive device 1 to the hand 100.

Further, the distance between the first curved portion 711 and the first base section 2 in a non-attached state is preferably, for example, in a range of about 10 through 40 mm, and more preferably in a range of about 20 through 30 mm.

Such a first curved portion 711 is connected to the first base section 2 using the second curved portion 712 and the third curved portion 713.

The second curved portion 712 functions as a connection section for connecting the fingertip side of the first curved portion 711 and an end portion on the fingertip side of the side surface 23 of the first base section 2 to each other. The second curved portion 712 is disposed between the thumb 110 and the index finger 101 in the attached state. Further, the second curved portion 712 curves so as to straddle the boundary between the palm 106 and the back-of-hand 105.

Meanwhile, the third curved portion 713 functions as a connection section for connecting the wrist side of the first curved portion 711 and an end portion on the wrist side of the side surface 23 of the first base section 2 to each other. The third curved portion 713 is disposed on an opposite side to the index finger 101 of the thumb 110, namely in the vicinity of a carpometacarpal joint 111 of the thumb 110 in the attached state. Further, the third curved portion 713 curves so as to straddle the boundary between the palm 106 side of the wrist and the back-of-hand 105 side of the wrist.

The second curved portion 712 and the third curved portion 713 can have contact with the hand 100, or can also be separated from the hand 100.

According to such a configuration, it is possible to prevent or inhibit the second curved portion 712 and the third curved portion 713 from hindering the action of the fingers, in particular the thumb 110 and the index finger 101, in the attached state.

The diameter D of the hook 71 is not particularly limited, but is preferably in a range of about 2 through 10 mm, and is more preferably in a range of about 4 through 8 mm. Thus, it is possible to prevent or inhibit the hook 71 from becoming an obstacle, and it is possible for the user to use the finger joint drive device 1 without an uncomfortable feeling.

Here, in the palm 106, when bending and stretching the finger, in particular the carpometacarpal joint (the third joint) of the thumb 110, a part nearer to the thumb 110 than the life line 200 mainly moves bounded by the life line 200. For example, when making the thumb 110 come closer to other fingers, the life line 200 forms a “valley” in the palm 106. In the finger joint drive device 1, the first curved portion 711 is disposed along the life line 200 in the attached state. Thus, even when bending and stretching the finger in the attached state, it is possible to prevent or inhibit the first curved portion 711 from hindering the bending and stretching of the finger. Therefore, in the attached state, a sufficient degree of freedom can be ensured.

Further, when changing from an unused state to the attached state, the thumb 110 is inserted in the loop formed of the hook 71 and the first base section 2. On this occasion, depending on the thickness of the hand 100 of the user, and the distance between the first curved portion 711 and the first base section 2, the base of the thumb 110 or the back-of-hand 105 is blocked by the loop described above, and the attachment of the finger joint drive device 1 becomes insufficient.

However, as described above, the hook 71 has the elasticity. Therefore, even in the case in which the base of the thumb 110 or the back-of-hand 105 is blocked by the loop, the hook 71 can deflect to deform, and thus, the attached state can be achieved. On this occasion, in the attached state, the first curved portion 711 biases the palm 106 toward the first base section 2 due to the restoring force of the hook 71. Therefore, the first curved portion 711 can surely clip the hand 100 together with the first base section 2. Further, since the hook 71 has the elasticity, it is possible to cope with the users different in thickness of the hand 100, and thus, the hook 71 is superior in general versatility.

It should be noted that the hook 71 can provide the advantage described above as long as the hook 71 includes a part lower in modulus of longitudinal elasticity than the first member.

Further, in the attachment mechanism 7, the first curved portion 711, the second curved portion 712, and the third curved portion 713 are made of a single continuous linear object, and are further formed integrally with each other. Thus, it is possible to easily manufacture the attachment mechanism 7 compared to the case of forming the first curved portion 711, the second curved portion 712, and the third curved portion 713 as separate members, and then bonding the separate members to each other.

It should be noted that the constituent material of the hook 71 is not particularly limited, and a variety of types of resin materials and a variety of types of metal materials can be used. As the resin materials described above, there can be cited, for example, polyolefin such as polyethylene, or polypropylene, ethylene-propylene copolymer, polyvinyl chloride, polystyrene, polyamide, polyimide, polycarbonate, poly-(4-methylpentene-1), ionomer, acrylic resin, polymethylmethacrylate, acrylonitrile-butadiene-styrene copolymer (ABS resin), acrylonitrile-styrene copolymer (AS resin), butadiene-styrene copolymer, fluorinated resin, epoxy resin, phenol resin, urea resin, melamine resin, silicone resin, polyurethane resin, and so on, copolymers, polymer blends, and polymer alloys having any one of these compounds as the primary constituent, and these materials can be used alone or in combination.

Further, the metal materials described above are not particularly limited, and there can be cited, for example, a variety of types of metal such as iron, nickel, stainless steel, copper, aluminum, and titanium, and alloys including any of these metals.

Then, an operation of the finger joint drive device 1 will be explained.

In the state shown in FIG. 2, the first base section 2 of the finger joint drive device 1 is attached to the back-of-hand 105, and the second base section 5 of the finger joint drive device 1 is attached to the proximal phalanx 102. On this occasion, as shown in FIGS. 1A and 1B, since the hand 100 is clipped by the first curved portion 711 and the first base section 2, it is possible to stably fix the finger joint drive device 1 to the hand 100.

Then, when the drive section 6A acts as described above in this state, it is possible to rotate the second link section 4 counterclockwise in the drawing as shown in FIG. 3. Thus, the proximal phalanx 102 of the index finger 101 is pressed toward an obliquely lower right direction in FIG. 3 together with the second base section 5. As a result, the metacarpophalangeal joint 108 of the index finger 101 bends.

Further, when the second link section 4 is rotated in the opposite direction to the above in the state shown in FIG. 3, the proximal phalanx 102 of the index finger 101 is pulled toward an obliquely upper left direction in the drawing together with the second base section 5. As a result, the metacarpophalangeal joint 108 of the index finger 101 stretches.

Further, when the metacarpophalangeal joint 108 bends (or stretches), it results that the second base section 5 gets away from (or closer to) the first base section 2. However, since the second link section 4 and the second base section 5 can relatively be displaced from each other as described above, the action of the second base section 5 getting away from (of closer to) the first base section 2 is promptly and smoothly performed. Thus, the metacarpophalangeal joint 108 can easily be bent, and the load on the index finger 101 can be reduced.

Further, it is possible for the user of the finger joint drive device 1 to bend a proximal interphalangeal joint 107 and a distal interphalangeal joint 109 of the index finger 101, the thumb 110, a middle finger, a third finger, and a little finger, which are not assisted by the finger joint drive device 1, independently of the metacarpophalangeal joint 108 of the index finger 101.

Further, in the finger joint drive device 1, the thickness of the whole of the device can be reduced compared to the case (see, e.g., JP-A-2002-345861) of adopting the configuration of bending and stretching the finger joint by sliding the member, which slides on, for example, the back-of-hand 105. Thus, when attaching the finger joint drive device 1 to use the finger joint drive device 1, it is possible to reduce the restriction applied to the action of the user.

It should be noted that since the first curved portion 711 of the attachment mechanism 7 is disposed along the life line 200, it is possible to prevent the bending and stretching of the index finger 101 from being hindered when bending and stretching the index finger 101. Further, according to the attachment mechanism 7, the degree of freedom of other fingers, in particular the thumb 110 can also be ensured.

Further, since the second link section 4 and the second base section 5 are relatively displaced from each other on the proximal phalanx 102 side of the index finger 101, the finger joint drive device 1 can be attached irrespective of the length of the index finger 101, and thus, high general versatility can be provided.

According to such an attachment mechanism 7, despite the simple configuration, the finger joint drive device 1 can stably be fixed to the hand, and at the same time, the degree of freedom of fingers can sufficiently be ensured.

Further, in the configuration (see, e.g., JP-A-2010-240285) in which the attachment mechanism covers the whole of the hand, since the whole of the attachment mechanism is driven, an extra drive force is required accordingly. In contrast, the attachment mechanism 7 is formed as a separate member from the part driven by the drive section 6A out of the finger joint drive device 1, and is not directly connected. Therefore, the finger can be bent and stretched with a relatively weak drive force.

### Second Embodiment

FIG. 6A is a diagram (a diagram viewed from the back side of a hand) showing a use state of a drive device (a second embodiment) according to the invention, and FIG. 6B is a diagram (a diagram viewed from the palm side of the hand) showing a use state of a finger joint drive device provided with an attachment mechanism shown in FIG. 6A.

The drive device according to the second embodiment of the invention will hereinafter be explained with reference to these drawings with a focus mainly on the differences from the embodiment described above, and the explanation regarding similar matters will be omitted.

The present embodiment is substantially the same as the first embodiment except the point that the configuration of the attachment mechanism is different.

As shown in FIG. 6A, in the attachment mechanism 7A, the both end portions of the hook 71 are connected to respective positions different from each other of the side surface 23 on the little finger 112 side of the first base section 2. Further, the second curved portion 712 and the third curved portion 713 are disposed on an opposite side of the little finger 112 to the third finger 113, namely on the outer side of a metacarpal (a fifth metacarpal) 114 of the little finger 112 in the attached state.

Further, as shown in FIG. 6B, the second curved portion 712 is longer than the second curved portion 712 in the first embodiment, and has a part 714 disposed on the palm 106. The part 714 extends from the little finger 112 side toward the index finger 101 so as to be arranged along a heart line 300 as close as possible.

Here, the heart line 300 is apart forming a “valley” in the palm 106 when bending the metacarpophalangeal joints of the index finger 101, the middle finger 114, the third finger 113, and the little finger 112. Since the part 714 is disposed on the heart line 300, even when bending and stretching the finger in the attached state, it is possible to prevent or inhibit the part 714 from hindering the bending and stretching of the finger. Therefore, in the attached state, a sufficient degree of freedom can be ensured.

### Third Embodiment

FIG. 7A is a diagram (a diagram viewed from the back side of a hand) showing a use state of a drive device (a third embodiment) according to the invention, and FIG. 6B is a diagram (a diagram viewed from the palm side of the hand) showing a use state of a finger joint drive device provided with an attachment mechanism shown in FIG. 7A.

The drive device according to the third embodiment of the invention will hereinafter be explained with reference to these drawings with a focus mainly on the differences from the embodiments described above, and the explanation regarding similar matters will be omitted.

The present embodiment is substantially the same as the first embodiment except the point that the configuration of the attachment mechanism is different.

As shown in FIGS. 7A and 7B, in the present embodiment, the hook 71 has the first curved portion 711, a pair of second curved portions 712, and a pair of third curved portions 713.

The second curved portions 712 are respectively disposed between the thumb 110 and the index finger 101, and on the opposite side of the little finger 112 to the third finger 113. Further, the second curved portions 712 are connected to the fingertip side of the first curved portion 711.

The third curved portions 713 are respectively disposed on the opposite side of the thumb 110 to the index finger 101, and on the opposite side of the little finger 112 to the third finger 113. Further, the third curved portions 713 are connected to the wrist side of the first curved portion 711.

According to such a configuration, the second curved portions 712 and the third curved portions 713 are respectively provided as pairs, and can therefore more stably fix the finger joint drive device 1 to the hand accordingly in the attached state.

Although the drive device according to the invention is hereinabove explained along the embodiments shown in the drawings, the invention is not limited to the embodiments, and each of the constituents of the drive device can be replaced with what can exert substantially the same function and has an arbitrary configuration. Further, it is also possible to add any constituents.

It should be noted that although in the embodiments described above, the third member is provided to the first member, the invention is not limited to this configuration, but the third member can be provided to the second member.

Further, although in the embodiments described above, the shim plate has contact with the third rotor in the vibrating section, the invention is not limited to this configuration, but the piezoelectric element can have contact with the third rotor.

Further, although in the embodiments described above, the attachment mechanism is formed of a single linear object, the invention is not limited to this configuration, but the linear object can partially be cut out, namely the linear object can end in the middle, for example.

Further, although the linear object has the elasticity throughout the entire length in the longitudinal direction, the invention is not limited to this configuration, but it is sufficient that the elasticity is provided to at least the connection section, for example. Further, even if the linear object does not have the elasticity, the advantage of the invention can be exerted.

Further, although in each of the embodiments, the drive device is attached to the hand, the invention is not limited to this configuration, but the drive device can be attached to a foot.

Further, although in each of the embodiments, the drive device is for driving the index finger, the invention is not limited to this configuration, but the drive device can be for driving, for example, the thumb, the middle finger, the third finger, or the little finger.

The entire disclosure of Japanese Patent Application No. 2014-056093, filed Mar. 19, 2014 is expressly incorporated by reference herein.

What is claimed is:

1. A drive device comprising:
a first member;
a second member rotatably disposed to the first member;
a rotating section adapted to rotate the second member; and
a third member provided to one of the first member and the second member, and having a hook.

2. The drive device according to claim 1, wherein
the rotating section has a vibrating section including a piezoelectric element, and a driven body having contact with the vibrating section and driven by the vibrating section.

3. The drive device according to claim 1, wherein
the hook has a first hook and a second hook.

4. The drive device according to claim 3, wherein
the first hook and the second hook are connected to each other in the third member.

5. The drive device according to claim 1, wherein
the drive device is to be disposed to a hand,
the first member is disposed on a back of the hand,
the second member is disposed on a finger of the hand,
the third member is provided to the first member, and
the hook has a contact section adapted to have contact with a palm of the hand.

6. The drive device according to claim 5, wherein
the hook has a part disposed between a thumb and an index finger of the hand.

7. The drive device according to claim 5, wherein
the hook has a part disposed on an opposite side of the thumb of the hand to the index finger of the hand.

8. The drive device according to claim 5, wherein
the hook has a first hook and a second hook,
the first hook is disposed between a thumb and an index finger of the hand, and
the second hook is disposed on an opposite side of the thumb of the hand to the index finger of the hand.

9. The drive device according to claim 5, wherein
the hook has a part disposed on an opposite side of a little finger of the hand to a third finger.

10. The drive device according to claim 5, wherein
the third member has a part arranged along a life line of the hand.

11. The drive device according to claim 1, wherein
the third member has a part lower in modulus of longitudinal elasticity than the first member.

12. The drive device according to claim 1, wherein
the third member is formed of a single continuous linear object.

13. The drive device according to claim 1, wherein
the hook has a first hook and a second hook, and
the first hook and the second hook are formed integrally with each other.
